(19) United States
(12) Patent Application Publication
(10) Pub. No.: US 2013/0231462 A1
(43) Pub. Date: Sep. 5, 2013

(54) ANTI-IMMUNE COMPLEX ANTIBODIES

(71) Applicant: BIO-RAD LABORATORIES, INC., Hercules, CA (US)

(72) Inventors: Steve Roth, Morgan Hill, CA (US); William Strong, El Cerrito, CA (US)

(73) Assignee: Bio-Rad Laboratories, Inc., Hercules, CA (US)

(21) Appl. No.: 13/776,403

(22) Filed: Feb. 25, 2013

Related U.S. Application Data
(60) Provisional application No. 61/603,685, filed on Feb. 27, 2012.

(51) Int. Cl. C07K 16/42

(52) U.S. Cl. CPC C07K 16/42
USPC 530/387.2

(57) ABSTRACT

Provided are methods and compositions for immunoassays with improved sensitivity and specificity. The presently disclosed anti-immune complex antibodies stabilize the interaction between a primary and secondary antibody, thereby allowing for more stringent wash conditions, less background, and stronger signal.

### CROSS REFERENCE TO RELATED APPLICATION

This application claims priority to U.S. Provisional Application No. 61/603,685, filed Feb. 27, 2012, the disclosure of which in incorporated by reference in its entirety.

### BACKGROUND OF THE INVENTION

Immunoassay and immuno separation techniques commonly rely on use of a primary antibody that specifically recognizes a target of interest and a secondary antibody that recognizes the primary antibody. The secondary antibody can be labeled, e.g., for indirect detection of the target, or attached to a matrix, e.g., for isolation of the target. The secondary antibody is typically generated to recognize a range of antibodies that may be used as primary antibodies, e.g., those from a certain species, or of a certain isotype. The relatively wide range of targets for the secondary antibody allows for more efficient use of expensive labeling or separation reagents—they are only attached to the secondary antibody, and not all of the primary antibodies.

The primary and secondary antibody system, however, does present technical challenges. Labeling or other conjugation of the secondary antibody can affect its binding properties. If the secondary antibody does not have high affinity for the primary antibody (e.g., there is a relatively high dissociation rate), wash conditions must be adjusted to be less stringent. This can result in high background and a loss of specificity. A high background can also make the assay less sensitive. That is, even if the primary antibody binds its target with high affinity and avidity, a less avid binding between the secondary and primary antibody can limit the efficacy of the entire procedure.

The presently disclosed anti-immune complex (AIC) antibodies can be used to stabilize the primary and secondary antibody interaction, resulting in more sensitive and specific target recognition.

### BRIEF SUMMARY OF THE INVENTION

Provided herein are anti-immune complex (AIC) antibodies that specifically recognize an immune complex, wherein the immune complex comprises a primary antibody bound by a secondary antibody, and optionally a bridge antigen. In some embodiments, a variable region of the AIC recognizes an epitope at the junction of the secondary antibody binding to the primary antibody (i.e., the AIC antibody does not significantly bind the primary or secondary antibody alone, or binds the immune complex with at least 5- or 10-fold higher affinity than the primary or secondary antibody alone). In some embodiments, the AIC comprises two variable regions that recognize an epitope at the junction of the secondary antibody binding to the primary antibody.

In some embodiments, the AIC is a bispecific antibody, and has two variable regions with different specificities. In some embodiments, the bispecific antibody comprises two distinct Fab or scFv polypeptides. In some embodiments, the bispecific antibody is a chimeric antibody. In some embodiments, the AIC is labeled (e.g., directly or indirectly bound to a detectable moiety).

In some embodiments, the AIC comprises a first variable region specific for a primary antibody and a second variable region specific for a secondary antibody. In some embodiments, the first variable region is specific for an Fc region epitope of the primary antibody. In some embodiments, the first variable region binds the primary antibody in a species-specific manner. In some embodiments, the secondary antibody binds the primary antibody in a species-specific manner. In some embodiments, the primary antibody is derived from a mammal. In some embodiments, the mammal is selected from mouse, rat, goat, rabbit, horse, donkey, pig, or human.

In some embodiments, the second variable region is specific for an Fv region epitope of the secondary antibody (e.g., an FR or C_{L} epitope). In some embodiments, the second variable region binds the secondary antibody in a species-specific manner (the secondary antibody is typically derived from a different species than the primary antibody). In some embodiments, the secondary antibody is derived from mouse, rat, goat, rabbit, horse, donkey, pig, or human.

In some embodiments, the AIC antibody comprises a first variable region specific for a primary antibody and a second variable region specific for a bridge antigen, wherein the primary antibody and bridge antigen are also specifically recognized by a secondary antibody. In some embodiments, the bridge antigen comprises an epitope from an Fc region, e.g., an Fc region epitope from the primary antibody. In some embodiments, the second variable region and the secondary antibody are specific for identical or similar epitopes. In some embodiments, the second variable region and the secondary antibody are specific for different epitopes on the bridge antigen. In some embodiments, the first and second variable regions of the AIC antibody are specific for the same epitope, while in some embodiments, the first and second variable regions of the AIC antibody are specific for different epitopes. In some embodiments, the secondary antibody is bispecific, and has two different variable regions, e.g., one specific for the primary antibody and one specific for the bridge antigen. In some embodiments, the secondary antibody is not bispecific.

Further provided is a stabilized immune complex comprising a primary antibody and a secondary antibody that specifically binds to the primary antibody to form an immune complex, and an anti-immune complex (AIC) antibody specifically bound to the immune complex. In some embodiments, the immune complex comprises a secondary antibody bound to two primary antibodies, and the stabilized immune complex comprises two AIC antibodies. In some embodiments, the Kd of the secondary antibody and primary antibody is at least 2-fold less in the stabilized immune complex than in the immune complex. In some embodiments, the Kd of the secondary antibody and primary antibody is at least 3-, 5-, 8-, 10- or 20-fold less in the stabilized immune complex than in the immune complex. That is, the AIC antibody reduces dissociation between the primary and secondary antibody. The AIC antibody thus extends the life-span of the immune complex to allow for delayed or multiple detection steps, or additional processing steps.

Provided are methods for stabilizing an immune complex, wherein the immune complex comprises a secondary antibody specifically bound to a primary antibody. In some embodiments, the method comprises: contacting a primary antibody with a secondary antibody specific for the primary antibody, thereby forming an immune complex; and contacting the immune complex with an AIC antibody as described herein. In some embodiments, the contacting steps are simultaneous, and in some embodiments, the contacting steps are sequential. In some embodiments, the method further comprises contacting the immune complex and AIC antibody with a bridge antigen, wherein the secondary antibody and AIC antibody are specific for the bridge antigen. In some embodiments, the method further comprises detecting the immune complex, e.g., with a detectable label on the target antigen, secondary antibody, AIC antibody, bridge antigen, and/or secondary binding molecule, e.g., a labeled streptavidin molecule. Where more than one component is labeled, the labels can be the same or different.

In some embodiments, the primary antibody is contacted with target antigen before being contacted with the secondary antibody and AIC antibody. In some embodiments, the immune complex is contacted with target antigen before being contacted with the AIC antibody. In some embodiments, the immune complex is contacted with target antigen after being contacted with the AIC antibody. In some embodiments, the target antigen, primary antibody, secondary antibody, AIC antibody, and bridge antigen, if present, are added to the same solution, essentially coming into contact simultaneously.

In some embodiments, the immune complex is stabilized by the AIC (+/−bridge antigen) in an immunoseparation or immunoprecipitation assay. In some embodiments, the immune complex is stabilized by the AIC (+/−bridge antigen) for detection in an immunodetection assay. In some embodiments, the immunodetection assay is selected from a Western blot, ELISA (direct, indirect, sandwich, quantitative, etc.), Southern blot (e.g., to detect target-conjugated nucleic acids, or distinctive nucleic acid moieties such as hairpins, modified, or non-naturally occurring nucleic acids), multiplex immunoassay (e.g., Bioplex), microsphere or magnetic bead-based immunoassay. In some embodiments, the immune complex is stabilized by the AIC during the immunoassay, e.g., during incubation of a Western blot membrane with antibodies. In some embodiments, the immunodetection assay is carried out in the absence of fixative agent, e.g., formaldehyde.

In some embodiments, the AIC antibody is labeled and detected. In some embodiments, the secondary antibody is labeled and detected. In some embodiments, the bridge antigen is labeled and detected. In some embodiments, a secondary adaptor molecule is labeled and detected, e.g., detection using an avidin-biotin complex. In some embodiments, the target antigen is labeled and detected. In some embodiments, two, three, four, or more components of the immunodetection assay are labeled and detected. In some embodiments, the labels of each component are the same. In some embodiments, the labels of each component are different.

Further provided are methods for generating (producing, making) an AIC antibody. In some embodiments, the method comprises introducing to an animal an immune complex comprising a secondary antibody specifically bound to a primary antibody, wherein said introducing results in an immunogenic response in the animal and production of antibodies specific for the immune complex; harvesting antibodies from the animal (e.g., the antibodies produced by the immunogenic response); selecting antibodies specific for the immune complex, thereby generating the AIC antibody. In some embodiments, the secondary antibody is covalently cross-linked (e.g., with a bifunctional cross-linker) to the primary antibody before the immune complex is introduced into the animal. In some embodiments, the method further comprises eliminating antibodies that specifically bind the primary or secondary antibody alone (i.e., negative selection). In some embodiments, the AIC antibody is a bispecific antibody. In some embodiments, the AIC antibody is not a bispecific antibody.

In some embodiments, the method for generating an AIC antibody comprises recombinantly expressing a first variable region specific for a primary antibody; and recombinantly expressing a second variable region specific for a secondary antibody or a bridge antigen, thereby forming an AIC antibody. In some embodiments, the first and second variable regions are expressed in the same cell. In some embodiments, the first variable region and second variable region are on the same polypeptide chain, e.g., a chimeric antibody. In some embodiments, the method further comprises associating the first variable region and second variable region, e.g., via a disulfide bond to form an F(ab′)2.

### BRIEF DESCRIPTION OF THE DRAWINGS

FIG. 1 shows an embodiment of the invention with a primary antibody (rabbit) bound by a labeled secondary antibody (anti-rabbit). The interaction is stabilized by the anti-immune complex antibody. In the example, the anti-immune complex (AIC) antibody binds the Fc region of the primary antibody and Fv region of the secondary antibody. The antibodies in FIG. 1 are depicted as tetramers with two heavy chains and two light chains.

FIG. 2 shows an embodiment of the invention with a primary antibody (rabbit) bound by an HRP-labeled secondary antibody (anti-rabbit). The secondary antibody also binds a bridge antigen (square), which is also bound by the AIC antibody (bottom right). In this embodiment, the AIC antibody stabilizes the primary and secondary antibody interaction via the bridge antigen. One of skill will appreciate that the secondary antibody and/or the anti-immune complex antibody can be bispecific or not. For example, the bridge antigen can be designed to carry epitopes similar or identical to those bound on the primary antibody. As with FIG. 1, the antibodies in FIG. 2 are depicted as tetramers with two heavy chains and two light chains.

### DETAILED DESCRIPTION OF THE INVENTION

### I. Introduction

Provided herein are anti-immune complex bispecific antibodies, immune complexes stabilized by such bispecific antibodies, methods of generating anti-immune complex bispecific antibodies, and immunoassays that use such bispecific antibodies, e.g., for improved sensitivity and/or specificity.

The presently described anti-immune complex antibodies can be used for any primary-secondary antibody immune complex, e.g., those with labeled secondary antibodies. The same anti-immune complex (AIC) antibody can be used with any assay involving the same secondary antibody specific for primary antibodies from a particular species. The AIC antibody can also be designed to be specific for more than one secondary-primary antibody pair. For example, the AIC antibody can be generated to recognize an epitope shared by all rodent primary antibodies, and an epitope shared by all anti-rodent secondary antibodies, or, e.g., all secondary antibodies from a particular species.

The anti-immune complex antibodies can increase specificity and decrease the dissociation rate of primary and secondary antibodies. Thus, background can be reduced and sensitivity increased in immunoassays (e.g., Western blotting, ELISAs, Southern blotting immunofluorescence, etc.). More stringent washing conditions can be used, thus reducing non-specific background while minimizing loss of specific signal from labeled secondary antibody interaction with the primary antibody. Moreover, because of the stronger interaction, less secondary antibody is required for immunoassays. Assay times can also be decreased, due to the strengthened interactions.

The AIC antibody can also be used to validate the specificity of a signal. For example, if both the secondary and AIC antibodies are labeled (with different labels), only signals with both labels would be considered specific.

### II. Definitions

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4^{th} ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The term “comprise” and variations thereof such as “comprises” and “comprising,” when preceding the recitation of a step or an element, are intended to mean that the addition of further steps or elements is optional and not excluded. Any methods, devices and materials similar or equivalent to those described herein can be used in the practice of this invention. The following definitions are provided to facilitate understanding of certain terms used frequently herein and are not meant to limit the scope of the present disclosure.

The term “immune complex” generally refers to the complex of a primary antibody and secondary antibody specific for the primary antibody. Typically, the secondary antibody is specific for a Fc region epitope on the primary antibody, and binds in a species specific manner, though this is not always the case. An example of a secondary antibody is an “anti-mouse” antibody, which will recognize primary antibodies derived from mice. The immune complex can further comprise a bridge antigen, as described in more detail herein. In some embodiments, where specified, the term “immune complex” can also refer to the complex of a Protein A, Protein G, or Protein A/G with a primary antibody.

An anti-immune complex (AIC) antibody is an antibody that specifically binds an immune complex, wherein the immune complex comprises both a primary antibody and secondary antibody, and optionally a bridge antigen bound to the secondary antibody. The anti-immune complex antibody can be bispecific, with a first variable region (antigen or analyte binding region) specific for the primary antibody, and a second variable region specific for the secondary antibody or bridge antigen. The anti-immune complex antibody can thus typically bind to each component of the immune complex alone, but is suited to bind the immune complex components when they are bound to one another. In the case where the immune complex is a complex of primary antibody with Protein A, Protein G, or Protein A/G, the AIC antibody has a first variable region specific for the primary antibody, and a second variable region specific for the Protein A, Protein G, or Protein A/G. One of skill will understand that the AIC antibody in this case will either lack the Protein A, Protein G, or Protein A/G binding site on its Fc region, or will be of an isotype, or from a species not recognized by Protein A, Protein G, or Protein A/G (whichever is included in the immune complex).

The term “bispecific antibody” refers to an antibody or fragments thereof that comprise two distinct variable regions (e.g., analyte recognition sites) specific for two distinct epitopes. A bispecific antibody can comprise two different Fv, Fab, or scFv regions (or any combination thereof) linked together with, e.g., a cross-linker, a disulfide bond, or amino acid linkages (e.g., in the case of a chimeric antibody). A bispecific antibody can also be generated in vivo by administering conjugated or cross-linked antigens (analytes) to an animal, e.g., as described in Wang et al. (2010) PLoS ONE 5:e10879. More typically, a bispecific antibody represents a man-made conjugate of two different antigen-binding sites. In some cases, the bispecific antibody is linked to an Fc region.

The term “primary antibody” will be understood by one of skill to refer to an antibody or fragment thereof that specifically binds to an analyte (e.g., substance, antigen, component) of interest. The primary antibody can further comprise a tag, e.g., for recognition by a secondary antibody or associated binding protein (e.g., GFP, biotin, or strepavidin), or to facilitate separation (e.g., a poly-His tag).

The term “secondary antibody” refers to an antibody that specifically binds to a primary antibody. A secondary antibody can be specific for the primary antibody (e.g., specific for primary antibodies derived from a particular species) or a tag on the primary antibody (e.g., GFP, biotin, or strepavidin). A secondary antibody can be bispecific, e.g., with one variable region specific for a primary antibody, and a second variable region specific for a bridge antigen.

The term “derived from,” with reference to an antibody, indicates that the antibody was originally isolated from cells of that type. For example, an antibody derived from a mouse is one that was originally obtained from a mouse, or mouse cell, but may have been further manipulated (e.g., labeled, recombinantly expressed, humanized, etc.). One of skill will understand that, in the case of a full length tetramer antibody, the Fc region of the antibody can have species-specific sequences that can be targeted for specific recognition, e.g., by a secondary antibody.

The term “antibody” refers to a polypeptide structure, e.g., an immunoglobulin, conjugate, or fragment thereof that retains antigen binding activity. The term includes but is not limited to polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompases conjugates, including but not limited to fusion proteins containing an immunoglobulin moiety (e.g., chimeric or bispecific antibodies or scFv's), and fragments, such as Fab, F(ab′)2, Fv, scFv, Fd, dAb and other compositions.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. The variable region contains the antigen-binding region of the antibody (or its functional equivalent) and is most critical in specificity and affinity of binding. See Paul, Fundamental Immunology (2003).

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies or those identified using phage display libraries (see, e.g., McCafferty et al., Nature 348:552-554 (1990)).

As used herein, the term “Fv” refers to a monovalent or bi-valent variable region fragment, and can encompass only the variable regions (e.g., V_{L} and/or V_{H}), as well as longer fragments, e.g., an Fab, Fab′ or F(ab′)2, which also includes C_{L} and/or C_{H}1. Unless otherwise specified, the term “Fc” refers to a heavy chain monomer or dimer comprising C_{H}1 and C_{H}2 regions.

A single chain Fv (scFv) refers to a polypeptide comprising a V_{L} and V_{H} joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

A “monoclonal antibody” refers to a clonal preparation of antibodies with a single binding specificity and affinity for a given epitope on an antigen. A “polyclonal antibody” refers to a preparation of antibodies that are raised against a single antigen, but with different binding specificities and affinities.

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, (1987) J Mol. Biol. 196, 901-917; Chothia et al. (1989) Nature 342, 877-883; Chothia et al. (1992) J. Mol. Biol. 227, 799-817; Al-Lazikani et al., J. Mol. Biol 1997, 273(4)). A helpful guide for locating CDRs using the Kabat system can be found at the website available at bioinf.org.uk/abs. Definitions of antigen combining sites are also described in the following: Ruiz et al. Nucleic Acids Res., 28, 219-221 (2000); and Lefranc Nucleic Acids Res. January 1; 29(1):207-9 (2001); MacCallum et al., J. Mol. Biol., 262: 732-745 (1996); and Martin et al, Proc. Natl Acad. Sci. USA, 86, 9268-9272 (1989); Martin, et al, Methods Enzymol., 203: 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

A “chimeric antibody” refers to an antibody in which (a) the constant region, or a portion thereof, is altered, replaced or exchanged so that the antigen binding site (variable region, CDR, or portion thereof) is linked to a constant region of a different or altered class, effector function and/or species; or (b) the variable region, or a portion thereof, is altered, replaced or exchanged with a variable region having a different or altered antigen specificity (e.g., CDR and framework regions from different species). Chimeric antibodies can include variable region fragments, e.g., a recombinant antibody comprising two Fab or Fv regions or an scFv. A chimeric can also, as indicated above, include an Fc region from a different source than the attached Fv regions. In some cases, the chimeric antibody includes chimerism within the Fv region. An example of such a chimeric antibody would be a humanized antibody where the FRs and CDRs are from different sources.

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polypeptide, polynucleotide, carbohydrate, lipid, chemical moiety, or combinations thereof (e.g., phosphorylated or glycosylated polypeptides, chromatin moieties, etc.). One of skill will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “bridge antigen” refers to an antigen that acts as a “bridge” or adaptor to link two or more components, e.g., two or more antibodies. A bridge antigen can comprise multiple epitopes, where each of the two or more antibodies recognize a distinct epitope. The bridge antigen can also comprise multiple instances of the same epitope, so that each of the two or more antibodies bind to identical or similar epitopes on the same antigen.

The terms “specific for,” “specifically binds,” and like terms refer to the binding of a molecule (e.g., antibody or antibody fragment) to a target (antigen, epitope, antibody target, etc.) with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds, or is specific for, a primary antibody will typically bind the primary antibody with at least a 2-fold greater affinity than a non-primary antibody target (e.g., an antibody from a different species or of a different isotype, or a non-antibody target).

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least 2/3 of the antibody targets in a solution (e.g., 75, 80, 85, 90, 91, 92, 93, 94, 95, 96, 97, 98, 99, or 100%). One of skill will recognize that some variability will arise depending on the method and/or threshold of determining binding.

As used herein, a first antibody, or an antigen-binding portion thereof, “competes” for binding to a target with a second antibody, or an antigen-binding portion thereof, when binding of the second antibody with the target is detectably decreased in the presence of the first antibody compared to the binding of the second antibody in the absence of the first antibody. The alternative, where the binding of the first antibody to the target is also detectably decreased in the presence of the second antibody, can, but need not be the case. That is, a second antibody can inhibit the binding of a first antibody to the target without that first antibody inhibiting the binding of the second antibody to the target. However, where each antibody detectably inhibits the binding of the other antibody to its cognate epitope or ligand, whether to the same, greater, or lesser extent, the antibodies are said to “cross-compete” with each other for binding of their respective epitope(s). Both competing and cross-competing antibodies are encompassed by the present invention. The term “competitor” antibody can be applied to the first or second antibody as can be determined by one of skill in the art. In some cases, the presence of the competitor antibody (e.g., the first antibody) reduces binding of the second antibody to the target by at least 10%, e.g., 20%, 30%, 40%, 50%, 60%, 70%, 80%, or more, e.g., so that binding of the second antibody to target is undetectable in the presence of the first (competitor) antibody.

The terms “label,” “detectable moiety,” and like terms refer to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target analyte. Any method known in the art for conjugating an antibody to the label may be employed, e.g., using methods described in Hermanson, Bioconjugate Techniques 1996, Academic Press, Inc., San Diego. The term “tag” can be used synonymously with the term “label,” but generally refers to an affinity-based moiety, e.g., a “His tag” for purification, or a “strepavidin tag” that interacts with biotin.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare signal strength in given conditions, e.g., in the presence of a test anti-immune complex antibody, in the absence of the test antibody (negative control), or in the presence of a known anti-immune complex antibody with a known affinity or a sample with covalently cross-linked primary and secondary antibodies (positive controls). One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term “nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

A variety of methods of specific DNA and RNA measurements that use nucleic acid hybridization techniques are known to those of skill in the art (see, Sambrook, Id.). Some methods involve electrophoretic separation (e.g., Southern blot for detecting DNA, and Northern blot for detecting RNA), but measurement of DNA and RNA can also be carried out in the absence of electrophoretic separation (e.g., quantitative PCR, dot blot, or array).

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encode a polypeptide are implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following amino acids are typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous,” with reference to a polynucleotide or polypeptide, indicates that the polynucleotide or polypeptide comprises two or more subsequences that are not found in the same relationship to each other in nature. For instance, a heterologous polynucleotide or polypeptide is typically recombinantly produced, having two or more sequences from unrelated genes arranged to make a new functional unit, e.g., a promoter from one source and a coding region from another source. Similarly, a heterologous protein indicates that the protein comprises two or more subsequences that are not found in the same relationship to each other in nature (e.g., a fusion protein).

### III. Anti-Immune Complex Antibodies

Provided herein anti-immune complex (AIC) antibodies, wherein the AIC specifically recognizes an immune complex comprising a primary antibody bound by a secondary antibody. In some embodiments, a variable region of the AIC recognizes an epitope at the junction of the secondary antibody binding to the primary antibody.

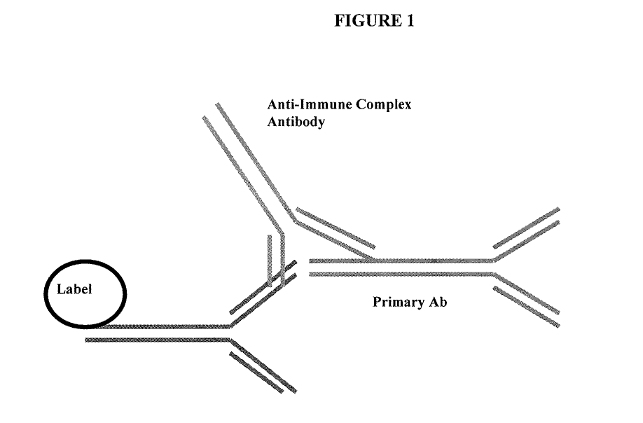

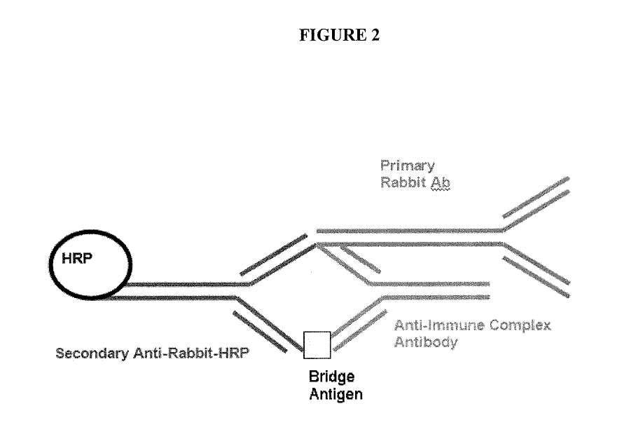

In some embodiments, the AIC antibody is a bispecific antibody comprising a first variable region specific for a primary antibody and a second variable region specific for a secondary antibody (see, e.g., FIG. 1). In some embodiments, the second variable region is specific for a bridge antigen instead of a secondary antibody (see, e.g., FIG. 2).

The terms primary and secondary antibody are familiar in the art. The primary antibody is typically selected to be specific for a target molecule of interest (antigen, analyte, etc., as described herein). The secondary antibody is typically labeled or immobilized, and is used to detect or bind to a primary antibody. Primary and secondary antibodies are used in a number of immunoassay formats to detect the presence of or amount of the target molecule.

The primary antibody can be a monoclonal or polyclonal antibody. Typically, the secondary antibody binds the Fc region of the primary antibody. In some embodiments, secondary antibody binding to the primary antibody is based on a species-specific epitope in the Fc region of the primary antibody. In some embodiments, the primary antibody is derived from a mammal. In some embodiments, the primary antibody is derived from mouse, rat, rabbit, goat, bovine, pig, donkey, sheep, guinea pig, chicken, human, or non-human primate, and the secondary antibody is specific for such a primary antibody.

In some embodiments, secondary antibody binding to the primary antibody is based on the isotype of the primary antibody, either alone, or in combination with the species the primary antibody was derived from. Thus, for example, the secondary antibody can be an anti-IgG (e.g., anti-IgG1, anti-IgG2, etc.), anti-IgM, anti-IgD, anti-IgA, or anti-IgE antibody.

In some embodiments, the primary antibody comprises a molecular tag, wherein the secondary antibody specifically binds the tag. For example, the tag could be a poly-histidine tag, a strepavidin or biotin tag, a GFP or other fluorescent protein tag, etc.

In some embodiments, the immune complex comprises a primary antibody, a secondary antibody specific for the primary antibody, and a bridge antigen, wherein the secondary antibody is also specific for the bridge antigen. In some embodiments, the bridge antigen includes an epitope that is similar to the epitope recognized by the secondary antibody on the primary antibody. For example, the bridge antigen can comprise at least part of an Fc region, e.g., from the same Fc region present in the primary antibody, or a shared Fc region epitope. In such cases, the secondary antibody can include two antigen binding regions (variable regions) specific for the same epitope. In some embodiments, the bridge antigen can include an epitope that is similar to the epitope recognized by the anti-immune complex antibody on the primary antibody. Thus, in such cases, the anti-immune complex antibody can include two antigen binding regions (variable regions) specific for the same epitope.

In some embodiments, the bridge antigen comprises multiple copies of identical (or substantially similar) epitopes, and the secondary antibody and AIC antibody recognize the identical or substantially similar epitopes on the bridge antigen. For example, the secondary antibody and AIC antibody can each be bivalent antibodies, but share antigen specificity in one of their variable regions. In some embodiments, the secondary antibody and AIC antibody are specific for different epitopes on the bridge antigen.

In some embodiments, the anti-immune complex (AIC) antibody comprises a first variable region specific for the primary antibody and a second variable region specific for the secondary antibody. In some embodiments, the first variable region is specific for a Fc region epitope on the primary antibody (e.g., the AIC specifically binds a site in the Fc region of the primary antibody). In some embodiments, the first variable region is specific for a species-specific epitope in the Fc region of the primary antibody. In some embodiments, the first variable region is specific for a primary antibody derived from a mammal. In some embodiments, the first variable region is specific for a primary antibody derived from a mouse, rat, rabbit, goat, bovine, pig, donkey, sheep, guinea pig, chicken, human, or non-human primate.

In some embodiments, the first variable region is isotype-specific, and binds a primary antibody that is IgG (e.g., IgG1, IgG2, etc.), IgM, IgD, IgA, or IgE isotype. In some embodiments, the first variable region is specific for the same target as the secondary antibody (e.g., both antibodies bind primary antibodies derived from rat), or in some cases, the same epitope. In some embodiments, the first variable region is specific for a primary antibody epitope outside the Fc region (e.g., a hinge or Fv region epitope, or a tag attached to the primary antibody). In some embodiments, the first variable region does not bind an Fc region epitope on the secondary antibody. In some embodiments, neither variable region of the AIC antibody binds an Fc region epitope on the secondary antibody.

In some embodiments, second variable region is specific for an Fv region epitope on the secondary antibody, e.g., an FR epitope. In some embodiments, the second variable region is specific for an epitope in FR1, FR2, FR3, FR4, C_{L}, or C_{H}1. In some embodiments, the second variable region is specific for a light chain epitope on the secondary antibody. In some embodiments, the second variable region is specific for a heavy chain epitope on the secondary antibody.

In some embodiments, the AIC antibody specifically binds an immune complex comprising a primary antibody and Protein A (with Protein A bound to the primary antibody). In some embodiments, the AIC antibody specifically binds an immune complex comprising a primary antibody and Protein G (with Protein G bound to the primary antibody). In some embodiments, the AIC antibody specifically binds an immune complex comprising a primary antibody and Protein A/G (with Protein A/G bound to the primary antibody). In such embodiments, the AIC antibody comprises a first variable region specific for the primary antibody and a second variable region specific for Protein A, Protein G, or Protein A/G (whichever is included in the immune complex). In some embodiments, the Protein A, Protein G, or Protein A/G is labeled. In some embodiments, the Protein A, Protein G, or Protein A/G is bound to a matrix, e.g., an affinity column, bead, or plastic petri dish or well. In some embodiments, the AIC antibody is labeled. In some embodiments, the AIC antibody is bound to a matrix.

In such embodiments, the AIC antibody typically is not recognized by the Protein A, Protein G, or Protein A/G. For example, the AIC antibody can be an antibody fragment or variant that lacks the Protein A, Protein G, or Protein A/G binding site on the Fc region of the AIC. The AIC can also be of an isotype that is not recognized (specifically bound) by Protein A, Protein G, or Protein A/G, or the AIC can be an antibody derived from a species that is not recognized by Protein A, Protein G, or Protein A/G. For example, Protein A, Protein G, and Protein A/G do not show significant binding to chicken antibodies, so the AIC antibody for an immune complex comprising a primary antibody and Protein A, Protein G, or Protein A/G can be derived from a chicken. Protein A does not show significant binding to antibodies from horse, human IgG3, human IgD, mouse IgG1, rat or sheep. Thus, where the immune complex comprises a primary antibody and Protein A, the AIC antibody can be derived from an antibody selected from the group consisting of horse, human IgG3, human IgD, mouse IgG1, rat or sheep. Protein G does not show significant binding to antibodies from cat, human IgM, human IgA, human IgE, or human IgD. Thus, where the immune complex comprises a primary antibody and Protein G, the AIC antibody can be derived from an antibody selected from the group consisting of cat, human IgM, human IgA, human IgE, and human IgD. One of skill will understand that the primary antibody typically will be one recognized by Protein A, Protein G, or Protein A/G, so that an immune complex is formed.

The presently described anti-immune complex antibodies typically bind to the immune complex with a binding affinity of about 10⁶, 10⁷, 10⁸, 10⁹, 10¹⁰, 10¹¹, or 10¹² M⁻¹( e.g., with a Kd in the micromolar (10⁻⁶), nanomolar (10⁻⁹), picomolar (10⁻¹²), or lower range). In some embodiments, the affinity of the first variable region for its epitope will be different than the affinity of the second variable region for its epitope. In some embodiments, the affinities will be similar, e.g., within one order of magnitude. In some embodiments, the affinity is expressed in terms of Kd, wherein

Kd=[antibody]×[target]/[antibody-target complex].

For example, the “antibody” in the above equation can refer to the anti-immune complex antibody, the “target” can refer to the immune complex, and the antibody-target complex can refer to a complex comprising the primary, secondary, and anti-immune complex antibodies. One of skill will understand that a higher affinity will correspond to a lower Kd (reduced dissociation).

The specificity of antibody binding can be defined in terms of the comparative dissociation constants (Kd) of the antibody for the target as compared to the dissociation constant with respect to the antibody and other materials in the environment or unrelated molecules in general. Typically, the Kd for the antibody with respect to the unrelated material will be at least 2-fold, 3-fold, 4-fold, 5-fold, 10-fold, 20-fold, 50-fold, 100-fold, 200-fold or higher than Kd with respect to the target.

A targeting moiety will typically bind with a Kd of less than about 1000 nM, e.g., less than 250, 100, 50, 20 or lower nM. In some embodiments, the Kd of the affinity agent is less than 15, 10, 5, or 1 nM. In some embodiments, the Kd is 1-100 nM, 0.1-50 nM, 0.1-10 nM, or 1-20 nM. The value of the dissociation constant (Kd) can be determined by well-known methods, and can be computed even for complex mixtures by methods as disclosed, e.g., in Caceci et al., Byte (1984) 9:340-362.

Affinity of an antibody, or any targeting agent, for a target can be determined according to methods known in the art, e.g., as reviewed in Ernst et al. Determination of Equilibrium Dissociation Constants, Therapeutic Monoclonal Antibodies (Wiley & Sons ed. 2009).

Quantitative ELISA, and similar array-based affinity methods can be used. ELISA (Enzyme linked immunosorbent signaling assay) is an antibody-based method. In some cases, an antibody specific for target of interest is affixed to a substrate, and contacted with a sample suspected of containing the target. The surface is then washed to remove unbound substances. Target binding can be detected in a variety of ways, e.g., using a second step with a labeled antibody, direct labeling of the target, or labeling of the primary antibody with a label that is detectable upon antigen binding. In some cases, the antigen is affixed to the substrate (e.g., using a substrate with high affinity for proteins, or a Strepavidin-biotin interaction) and detected using a labeled antibody (or other targeting moiety). Several permutations of the original ELISA methods have been developed and are known in the art (see Lequin (2005) Clin. Chem. 51:2415-18 for a review).

The Kd, Kon, and Koff can also be determined using surface plasmon resonance (SPR). SPR techniques are reviewed, e.g., in Hahnfeld et al. Determination of Kinetic Data Using SPR Biosensors, Molecular Diagnosis of Infectious Diseases (2004). In a typical SPR experiment, one interactant (target or targeting agent) is immobilized on an SPR-active, gold-coated glass slide in a flow cell, and a sample containing the other interactant is introduced to flow across the surface. When light of a given frequency is shined on the surface, the changes to the optical reflectivity of the gold indicate binding, and the kinetics of binding.

Binding affinity can also be determined by anchoring a biotinylated interactant to a streptaviden (SA) sensor chip. The other interactant is then contacted with the chip and detected, e.g., as described in Abdessamad et al. (2002) Nuc. Acids Res. 30:e45.

Binding affinity can also be determined using comparative methods. For example, a set of components with known affinities can be compared to the test components (i.e., antibody and target) under various conditions, e.g., wash conditions of various stringencies.

### IV. Methods of Generating Antibodies

For preparation and use of antibodies as described herein, e.g., monoclonal, recombinant, and/or bispecifc antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)).

In some embodiments, an anti-immune complex antibody is generated by introducing an immune complex of interest (i.e., a secondary antibody binding a primary antibody, and optionally binding a bridge antigen) into an animal. Such methods are described e.g., in Coligan, Current Protocols in Immunology (1991); Harlow & Lane, Antibodies, A Laboratory Manual (1988); and Wang et al. (2011) PLoS ONE 5:e10879. The immune complex can be stabilized for in vivo administration by chemical cross-linking. The animal is typically a mammal, such as a mouse, rat, rabbit, goat, horse, pig, etc., such that the animal mounts an immune response against the immune complex. Antibodies generated by the immune response can then be used as the basis for generating a monoclonal antibody using known methods.

Techniques for the production of single chain antibodies or recombinant antibodies are known and can be used to produce antibodies, e.g., anti-immune complex antibodies as described herein. Also, transgenic mice, or other organisms such as other mammals, can be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

Bispecific antibodies (which recognize two different antigens) can be generated as described, e.g., in Doppalapudi et al. (2010) Proc Natl Acad Sci 107:22611, which describes a method for rapid, chemical linkage of distinct antibody segments. Additional methods of generating bispecific antibodies are described, e.g., in WO93/08829; Traunecker et al., EMBO J. 10:3655-3659 (1991); and Suresh et al., Methods in Enzymology 121:210 (1986)). Antibodies can also be heteroconjugates, e.g., two covalently joined antibodies (see, e.g., U.S. Pat. No. 4,676,980 , WO91/00360; WO 92/200373; and EP 03089).

Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers. In embodiments in which an antibody comprises both a V_{H} and V_{L} region, the V_{H} and V_{L} regions may be expressed using a single vector, e.g., in a di-cistronic expression unit, or under the control of different promoters. In other embodiments, the V_{H} and V_{L} region may be expressed using separate vectors.

An antibody as described herein can also be produced in various formats, including as a Fab, a Fab′, a F(ab′)₂, a scFv, or a dAB (diabody). The antibody fragments can be obtained by a variety of methods, including, digestion of an intact antibody with an enzyme, such as pepsin (to generate (Fab′)₂ fragments) or papain (to generate Fab fragments); or de novo synthesis. Antibody fragments can also be synthesized using recombinant DNA methodology. See, e.g., Fundamental Immunology (Paul ed., 2003); Bird, et al., Science 242:423 (1988); and Huston, et al., Proc. Natl. Acad. Sci. USA 85:5879 (1988).

In some cases, the antibody or antibody fragment can be conjugated to another molecule, e.g., polyethylene glycol (PEGylation), for improved stability. Examples of PEGylation of antibody fragments are provided in Knight et al. Platelets 15:409, 2004 (for abciximab); Pedley et al., Br. J. Cancer 70:1126, 1994 (for an anti-CEA antibody); Chapman et al., Nature Biotech. 17:780, 1999; and Humphreys, et al., Protein Eng. Des. 20: 227, 2007). The antibody or antibody fragment can also be labeled or tagged as described below.

### V. Labels

The antibodies, bridge antigens, and target antigens described herein can be conjugated or otherwise associated with a detectable label. The association can be direct e.g., a covalent bond, or indirect, e.g., using a secondary binding agent, chelator, or linker. The terms “detectable agent,” “detectable label,” “detectable moiety,” “label,” “imaging agent,” and like terms are used synonymously herein. In some embodiments, the AIC antibody is labeled. In some embodiments, the secondary antibody is labeled. In some embodiments, the AIC and secondary antibodies are labeled, e.g., with the same or with different labels. In some embodiments, the bridge antigen and AIC antibody and/or secondary antibody are labeled. In some embodiments, the target antigen and AIC antibody and/or secondary antibody are labeled.

In some embodiments, the label can include an optical agent such as a fluorescent agent, phosphorescent agent, chemiluminescent agent, etc. Numerous agents (e.g., dyes, probes, labels, or indicators) are known in the art and can be used in the present invention. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives.

The presently disclosed antibodies can be used for immunoassays, e.g., Western blots, ELISAs, Southern (e.g., to detect biotinylated nucleic acid amplification products, or other distinctive nucleic acid moieties), FACS, immunoprecipitation, immunohistochemistry, immunofluorescence (e.g., using cells or tissue from a cell line or patient sample). In some embodiments, the immunoassay is multiplex, or carried out automatically, e.g., using Bio-Plex® or similar systems. In some embodiments, cells or cellular material used in the immunoassay is fixed. In some embodiments, cells or cellular material is not fixed.

A radioisotope can be used as a label, and can include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹⁶⁶Ho, ¹²³I, ¹²⁴I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃triamine (cyclic or linear). In other embodiments, the agents can include DOTA and its various analogs with ¹¹¹In, ¹⁷⁷Lu, ¹⁵³Sm, ^{88/90}Y, ^{62/64/67}Cu, or ^{67/68}Ga. I

In some embodiments, the antibody (e.g., the secondary or AIC antibody) or antigen (e.g., bridge or target antigen) can be associated with a secondary binding ligand or to an enzyme (an enzyme tag) that will generate a colored product upon contact with a chromogenic substrate. Examples of suitable enzymes include urease, alkaline phosphatase, (horseradish) hydrogen peroxidase (HRP) and glucose oxidase. Secondary binding ligands include, e.g., biotin and avidin or streptavidin, as known in the art. In some embodiments, the label is a fluorescent protein sequence, and can be recombinantly combined with the antibody polypeptide sequence.

In some embodiments, the antibody or antigen is labeled so as to amplify the signal, e.g., with an avidin-biotin complex (ABC) labeling system as described in WO2012/122121. In some embodiments, the secondary and/or AIC antibody is labeled with biotin. Biotin (and like molecules) is bound by streptavidin (and like molecules), which can be labeled, and detected with a biotinylated antibody specific for the streptavidin or its label. The second biotinylated antibody can then in turn provide multiple biotin binding sites, which results in amplified signal. One of skill will appreciate that the ABC system can be varied according to the assay, with several variations described in WO2012/122121.

Techniques for conjugating detectable agents to antibodies and other molecules are well known and antibody labeling kits are commercially available from dozens of sources (e.g., Invitrogen, Pierce, Sigma Aldrich, Biotium, Jackson Immunoresearch, etc.). A review of common protein labeling techniques can be found in Biochemical Techniques: Theory and Practice (1987).

Antibodies and targets are generally labeled in an area that does not interfere with antibody-target binding, or with stability of the immune complex. In some embodiments, the detectable moiety is attached to the constant region, or outside the CDRs in the variable region. One of skill in the art will recognize that the optimal position for attachment may be located elsewhere on the antibody, so the position of the detectable moiety can be adjusted accordingly. In the case of a labeled antigen, one of skill will appreciate that the label should not interfere with the epitope recognized by the antibody. In some embodiments, the ability of the antibody to associate with the epitope is compared before and after attachment to the detectable moiety to ensure that the attachment does not unduly disrupt binding.

### VI. Immunoassays and Antibody-Based Techniques

The AIC antibodies described herein can be used with any antibody-based assay or separation procedure where a primary and secondary antibody can be employed. One of skill will recognize that the present compositions and methods can be practiced with any combination of primary antibody and secondary antibody, and multiple combinations, e.g., where the AIC antibody is specific for more than one primary antibody (e.g., all mouse primary antibodies) and/or more than one secondary antibody (e.g., all rabbit secondary antibodies).

The AIC antibodies described herein provide a number of advantages for immunoassays and immunoseparation. The immune complex is stabilized by the AIC, so that the time for detecting (or washing, analyzing, processing, etc.) is extended. This allows for multiple reads, e.g., for multiple comparisons, additional processing steps, etc. Current methods rely on formaldehyde or like chemicals to “fix” a detectable signal, and extend the time available for detection. Formaldehyde has an unpleasant smell, can have adverse effects on the assay components (e.g., enzymes), and can be harmful to the user.

Examples of immunoassays include, enzyme linked immunoabsorbent assay (ELISA), fluorescent immunosorbent assay (FIA), immunohistochemistry, free or ambient analyte immunoassays, microsphere-based immunoassays, chemical linked immunosorbent assay (CLIA), radio-immuno assay (RIA), flow cytometry (e.g., fluorescence activated cell sorting or FACS), Western blot, Southern blot, and immunoblotting. Additional applicable immunotechniques include competitive and non-competitive assay systems, e.g., “sandwich” immunoassays, immunoprecipitation assays, precipitin reactions, immunodiffusion assays, immunoradiometric assays, fluorescent immunoassays, etc. Immunoassays can be multiplex, with multiple simultaneous or sequential assays, or carried out automatically, e.g., using Bio-Plex® or similar systems. For a review of immunoassays for which the presently described AICs can be used, see, e.g., The Immunoassay Handbook, David Wild, 3^{rd} ed., Stockton Press, New York, 2005; Ausubel et al, eds, 1994, Current Protocols in Molecular Biology, Vol. 1, John Wiley & Sons, Inc., New York.

Western blotting is usually used to detect the presence or relative amount of a given target. The technique generally comprises preparing protein samples, electrophoresis of the protein samples in a polyacrylamide gel (e.g., 8%-20% SDS-PAGE), transferring the proteins from the polyacrylamide gel to a membrane such as nitrocellulose, PVDF or nylon, blocking the membrane in blocking solution (e.g., PBS with 3% BSA or non-fat milk), washing the membrane, contacting the membrane with primary antibody diluted in blocking buffer, washing the membrane in washing buffer, incubating the membrane with a labeled secondary antibody diluted in blocking buffer, washing the membrane in wash buffer, and detecting the presence or amount of the target by detecting the presence or amount of the label.

ELISAs, in basic form, comprise preparing a target antigen, coating the wells of a multiwell microtiter plate with the antigen, adding primary antibody, and incubating for a period of time, followed by addition of labeled secondary antibody. One of skill in the art would be knowledgeable as to other variations of ELISAs where the present AIC antibodies will be useful to stabilize the primary and secondary antibody interaction.

The presently described AIC antibodies can be used to increase signal strength, and improve specificity in immunodetection assays. For example, the AIC antibody and the secondary antibody can both be detectably labeled, either with the same or different labels. In some embodiments, the AIC antibody is labeled with a different label than the secondary antibody, e.g., to ensure that only the intended primary-secondary antibody complex is detected when both labels are detected. In some embodiments, the AIC antibody is labeled with the same label as the secondary antibody, e.g., to improve sensitivity in assays where the primary-secondary complex is expected to be rare. In some embodiments, the bridge antigen is labeled, either with the same or a different label than the secondary, to similar effect as labeling the AIC antibody. Use of labeled bridge antigens in particular allows for flexibility at low cost if multiple labels are desired. Signal amplification can also be achieved using the ABC system described above.

The present AIC antibodies allow these assays (and others) to be streamlined by enhancing the strength of the association between the secondary and primary antibody. The incubations can be simultaneous, and the washing steps can be more stringent (e.g., higher % detergent or higher temperature). Due to the stabilized immune complex, the assay can produce more sensitive and specific signal even with more stringent conditions and shorter incubations.

Immunoprecipitation and immunoseparation protocols can comprise contacting a sample (e.g., cell lysate) with primary antibody specific for the desired target in the sample, incubating for a period of time (e.g., 1-4 hours at 4° C.), adding secondary antibody-coated sepharose beads (or other support matrix) to the mixture and incubating again, washing the beads, and resuspending the beads in an SDS/sample buffer or elution buffer. Again, one of skill will be familiar with variations of the technique, e.g., use of magnetic beads or chromatography for immunoseparation. As with the immunodetection assays above, the AIC antibodies can be used to streamline the process, while improving the sensitivity and specificity of the target separation.

### VII. Kits

Further provided are kits for immunodetection or immunoseparation, wherein the kit comprises an AIC antibody as described herein. In some embodiments, the AIC specifically recognizes an immune complex comprising a primary antibody bound by a secondary antibody. In some embodiments, the kit includes an AIC antibody comprising a first variable region specific for a primary antibody, e.g., primary antibodies derived from a certain species (e.g., mouse, rat, goat, rabbit, horse, donkey, pig, or human), and a second variable region specific for a secondary antibody. In some embodiments, the secondary antibody is specific for primary antibodies of the same species as that recognized by the first variable region. In some embodiments, the kit further combines the secondary antibody. In some embodiments, the second variable region is specific for secondary antibodies derived from a certain species, wherein the primary and secondary antibodies are derived from different species.

In some embodiments, the kit includes an AIC antibody comprising a first variable region specific for a primary antibody, e.g., primary antibodies derived from a certain species (e.g., mouse, rat, goat, rabbit, horse, donkey, pig, or human), and a second variable region specific for a bridge antigen. In some embodiments, the bridge antigen includes at least a part of an Fc region, e.g., an Fc region epitope found on a primary antibody. In some embodiments, the kit includes a bridge antigen. In some embodiments, the kit further includes a secondary antibody that specifically binds the bridge antigen and the primary antibody.

In some embodiments, where the kit includes a secondary antibody, the secondary antibody is labeled. In some embodiments, the kit includes reagents for labeling an antibody. In some embodiments, the AIC antibody is labeled. In some embodiments, the secondary and AIC antibodies are labeled, e.g., with different labels.

In some embodiments, the kit includes supplies and reagents for carrying out an immunoassay or immunoseparation, such as blots (e.g., nylon or nitrocellulose), ELISA plates, buffer stock solutions, markers and/or controls, chromatography supplies, size or charge separation columns, etc.

The kit will also typically include instructions for use, or direction to an outside source of instruction such as a website.

It is understood that the examples and embodiments described herein are for illustrative purposes only and that various modifications or changes in light thereof will be suggested to persons skilled in the art and are to be included within the spirit and purview of this application and scope of the appended claims. All patents, patent applications, internet sources, and other published reference materials cited in this specification are incorporated herein by reference in their entireties. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

1. An isolated bispecific antibody comprising
a first variable region specific for a primary antibody, and
a second variable region specific for a secondary antibody or a bridge antigen.

2. The bispecific antibody of claim 1, wherein the first variable region is specific for an Fc region epitope of the primary antibody.

3. The bispecific antibody of claim 1, wherein the first variable region binds the primary antibody in a species-specific manner.

4. (canceled)

5. The bispecific antibody of claim 1, wherein the primary antibody is derived from mouse, rat, goat, rabbit, horse, donkey, pig, or human.

6. The bispecific antibody of claim 1, wherein the second variable region is specific for an Fv region epitope of the secondary antibody.

7. The bispecific antibody of claim 1, wherein the second variable region binds the secondary antibody in a species-specific manner.

8. (canceled)

9. The bispecific antibody of claim 1, wherein the secondary antibody is derived from mouse, rat, goat, rabbit, horse, donkey, pig, or human.

10. The bispecific antibody of claim 1, wherein the second variable region and the secondary antibody are specific for a bridge antigen.

11. The bispecific antibody of claim 1, wherein the bispecific antibody comprises two distinct Fab or scFv polypeptides.

12-13. (canceled)

14. A method of producing an anti-immune complex (AIC) antibody, wherein the AIC antibody is specific for an immune complex comprising a secondary antibody bound to a primary antibody, the method comprising
introducing to an animal the immune complex, wherein said introducing results in an immunogenic response in the animal;
harvesting antibodies generated by the immunogenic response in the animal;
selecting antibodies that are specific for the immune complex, thereby producing the AIC antibody.

15. The method of claim 14, wherein the AIC antibody is a bispecific antibody.

16. A method for stabilizing an immune complex, the method comprising
(i) contacting a primary antibody with a secondary antibody specific for the primary antibody, thereby forming the immune complex; and
(ii) contacting the immune complex with the bispecific antibody of claim 1, thereby stabilizing the immune complex.

17-20. (canceled)

21. A method for stabilizing an immune complex, the method comprising
i) contacting a primary antibody with a secondary antibody specific for the primary antibody, thereby forming an immune complex; and
ii) contacting the immune complex with an anti-immune complex (AIC) antibody that specifically binds the immune complex, thereby stabilizing the immune complex.

22. The method of claim 21, wherein the AIC antibody is a bispecific antibody, wherein the bispecific antibody comprises a first variable region specific for the primary antibody and a second variable region specific for the secondary antibody.

23-32. (canceled)

33. A method for stabilizing an immune complex, the method comprising
i) contacting a primary antibody with a secondary antibody specific for the primary antibody, thereby forming an immune complex; and
ii) contacting the immune complex with a bispecific antibody and a bridge antigen, wherein the bispecific antibody comprises a first variable region specific for the primary antibody and a second variable region specific for the bridge antigen, and the secondary antibody is specific for the bridge antigen, thereby stabilizing the immune complex.

34-38. (canceled)

39. The method of claim 33, wherein the immune complex is used to determine the presence or amount of a target in an immunoassay, and wherein the primary antibody specifically recognizes the target.

40. The method of claim 39, wherein the immunoassay is a Western blot.

41. A stabilized immune complex comprising
a primary antibody,
a secondary antibody specifically bound to the primary antibody, thereby forming an immune complex, and
an anti-immune complex (AIC) antibody specifically bound to the immune complex.

42. The stabilized immune complex of claim 41, wherein the AIC antibody is a bispecific antibody comprising a first variable region specific for the primary antibody and a second variable region specific for the secondary antibody.

43. The stabilized immune complex of claim 41, wherein the Kd of the stabilized immune complex is at least 5-fold lower than the Kd of the immune complex lacking the AIC antibody.

44-45. (canceled)
